(19) United States
(12) Patent Application Publication
(10) Pub. No.: US 2014/0112969 A1
(43) Pub. Date: Apr. 24, 2014

(54) DYNAMIC LOADING OF A THERAPEUTIC FLUID

(71) Applicant: SpineSmith Partners, L.P., Austin, TX (US)

(72) Inventor: Theodore Sand, Austin, TX (US)

(73) Assignee: SpineSmith Partners, L.P., Austin, TX (US)

(21) Appl. No.: 14/061,628

(22) Filed: Oct. 23, 2013

Related U.S. Application Data
(60) Provisional application No. 61/717,482, filed on Oct. 23, 2012.

(51) Int. Cl. A61L 31/16; A61L 31/04; A61L 31/00

(52) U.S. Cl. CPC A61L 31/16; A61L 31/005; A61L 31/047; A61L 31/046
USPC 424/423; 424/93.7; 514/7.6; 514/13.7

(57) ABSTRACT

The invention is directed to a method for therapeutic repair of an injury to bone or tissue comprising providing a structural component, wherein the structural component repairs the injury; and providing a therapeutic fluid, wherein the therapeutic fluid is located within the structural component.

### CROSS-REFERENCES TO RELATED APPLICATIONS

This Application claims the benefit under 35 U.S.C. §119(e) of U.S. Provisional Patent Application No. 61/717,482 filed Oct. 23, 2012 which is incorporated herein by reference in its entirety as if fully set forth herein.

### BACKGROUND OF THE INVENTION

It is well known that the use of interference screws in surgical repairs of orthopedic injuries like an anterior cruciate ligament (ACL) repair or other ligamentous or tendonous repairs requiring anchoring devices can result in localized trauma to the bone in which the anchor or interference screw is placed. Trauma to the bone through which a screw is inserted in an ACL repair occurs due to the extremely tight fit of the screw threads as the screw is advanced through the tissue to tightly seal the tendons that are used to provide stability to the joint. There are issues related to the stability of highly compressed bone, due to potential necrosis of the compressed bone. Necrosis of the adjacent bony tissue could result in a reduction in the stability of the surgical repair, leading to a failure and a need for revision. Once an interference screw has been set in place, it is difficult or impossible to provide contact of the compressed bony tissue with a therapeutic fluid containing cells and/or other components known to provide a therapeutic benefit. The lack of access and the difficulty of placing therapeutic fluids in contact with the bony tissue most damaged suggest a need to make the therapeutic fluids available while the device is being implanted. Such an approach is termed “dynamic loading”.

It is common to use fluids in washing out wounds or other surgical repairs during a surgical procedure or a treatment. Such solutions might include saline or Lactated Ringer's. Simple flushing is accomplished by using a dispensing container, like a large volume syringe, and directing the outlet at the site to be flushed. The use of cell-containing fluids or fluids composed of proteins (i.e., fibrinogen and thrombin, among others) has been applied by spraying or spreading the therapeutic fluids on the open wound. Specially designed spray tips (available from a company like Micromedic) have been adapted to coat open wounds with therapeutic fluids containing cells (i.e., mesenchymal stem cells, and/or other progenitor cells). However, these devices and procedures are not applicable to treating the compression of adjacent bony tissue when performing an ACL repair with a standard interference screw. The only option would be to spray the “tunnel” in which the interference screw will be inserted. This approach does not guarantee that any therapeutic fluid will be present upon screw insertion.

### SUMMARY OF THE INVENTION

An embodiment of the invention is directed to a method for therapeutic repair of an injury to bone or tissue comprising providing a structural component, wherein the structural component repairs the injury; and providing a therapeutic fluid, wherein the therapeutic fluid is located within the structural component.

### DETAILED DESCRIPTION OF EXEMPLARY EMBODIMENTS

In an embodiment of the invention, the inventive approach of “dynamic loading” results in the concurrent provision of a therapeutic fluid while the highly compressive forces are being exerted during the insertion of, for example, an interference screw. In the case of an ACL repair, the therapeutic fluid is present as the bony tissue is being compressed, thereby ensuring that the compressed tissue is exposed to the therapeutic fluid while the bony tissue is being repaired. Dynamic loading further ensures that there is sufficient contact time between the therapeutic fluid and the site at which the repair is being performed in order to potentiate the therapeutic benefit of the therapeutic fluid. The approach of dynamic loading is compatible with therapeutic fluids that might contain therapeutic cells (i.e., stem cells) and proteins or proteins alone or cells alone.

An embodiment of the invention provides an inventive procedure of “dynamic loading” comprising the steps of providing a therapeutic fluid while also performing an orthopedic repair in which compression of bony tissue is required in order to obtain a therapeutic benefit and allow the patient to recover. For example, the traditional way to repair a torn anterior cruciate ligament is to use tendon tissue to line a tunnel bored into the tibial head and anchored in the femoral head. An interference screw is then inserted into and seated in the tibial head tunnel, thereby compressing the tendons against the bony tissue. The greater the compression, achieved by using a larger diameter interference screw, in theory, the tighter the hold and the more secure the ACL repair. However, the type of bone present along the length of the tunnel is not uniform, so that the holding ability of the bone along the tunnel is not consistent. Furthermore, over-compression of the bony tissue along the tunnel could lead to necrosis.

In an embodiment of the invention, dynamic loading is adapted to a traditional interference screw repair by use of a fenestrated and cannulated screw. The insertion tool comprises a driver that advances the screw position, while pressure is placed on a reservoir containing the therapeutic fluid that forces fluid into the cannulated bore of the screw via a thin tubing. As the screw is advanced in the bony tissue, therapeutic fluid floods the interior of the screw bore and moves into the compressing bony tissue. The tubing from the reservoir fits within the shaft of the driver and the engagement of the driver and the screw will be such that the tubing rotates within the screw head so as not to interfere as the screw is advanced. Due to the need to not compromise the structural integrity of the threads on the screw, cannulation and fenestration of the screw needs to be very carefully achieved.

Alternate designs for achieving the therapeutic repair of an interference screw also can be adapted to provide dynamic loading of therapeutic fluids. One device design would require the insertion of the device via the use of a mandrel and a tensioning tool to maintain the correct tension of the tendons during the compressive phase of the ACL repair. The same approach for using a driver when using a traditional screw for repair also can be adapted when using a mandrel. In the case of a mandrel, it would be cannulated and fenestrated along the length of the mandrel. Flow of therapeutic fluid from the attached reservoir to the mandrel is achieved by the use of a thin tubing. As the mandrel is advanced fluid is flowing through the fenestrations outward toward the walls of the tunnel as compression of the bony tissue in the tunnel occurs. The proximity of the therapeutic fluid to the zone of compression provides the greatest potential of the therapeutic fluid contributing to the surgical repair by reducing deleterious outcomes like necrotic tissue.

The composition of the therapeutic fluid comprises cells, like mesenchymal stem cells, and proteins, like fibrinogen and growth factors. Other components present in the therapeutic fluid can include fluids like platelet-rich plasma (PRP), platelet poor plasma (PPP), or concentrated forms of PRP and PPP. It also is possible to include recombinant proteins like rhBMP (recombinant human bone morphogenic protein). Consequently, the composition of the therapeutic fluid that is used in dynamic loading can be varied and adapted to the specific repair being performed.

### WORKING EXAMPLE

### Example 1

### Dynamic Loading of an Interference Screw

Fresh human bone marrow was taken up in a 15 mL syringe and then clotted in the syringe by addition of 10% CaCl₂ and 1000 Units/mL bovine thrombin promptly followed by thorough mixing in the syringe. A luer-lock valve and tubing was attached to the end of the syringe and the bone marrow clot could be expressed out of the syringe. The marrow maintained the viscous consistency of a clot after leaving the syringe. Cell viability was measured by the LIVE/DEAD® Viability/Cytotoxicity Kit (Molecular Probes). The kit contains Calcein AM (CAM) and Ethidium-1 (EthD-1) stains. CAM stains viable cells green by permeating live cells and undergoing enzymatic conversion to fluoresce. EthD-1 enters damaged cell membranes and fluoresces red when it binds to nucleic acids but it is excluded by intact cells.

Bone marrow was prepared with the LIVE/DEAD® stain at concentrations of 10□M CAM and 10 μM EthD-1. The marrow was clotted inside a 15 mL syringe. The syringe was connected by the luer-lock valve and tubing to an interference screw. The clotted bone marrow could be dynamically loaded through the luer-lock connector and tubing to fill the interference screw. The interference screws can be removed from the tubing and the marrow clot would remain inside the screws.

The interference screws loaded with stained and clotted bone marrow concentrate were examined by confocal microscopy. The interference screws were made of a transparent polymer so the confocal microscope could be used to image stained cells that had been dynamically loaded inside the screws.

Table 1 summarizes the ImageJ analysis of the images of the stained cells inside the interference screw. The areas inside the screws with stained live cells consistently showed higher greyscale values than the same areas inside the screws stained for dead cells. The higher greyscale values show that a higher amount fluorescence was produced by live cells in the same region compared to dead cells. The ratios of live/dead cells supports the dynamic delivery of cells into an interference screw in such a manner as to retain viable cells.

TABLE 1
Mean Gray Scale Intensity Value

| | Live Cells | Dead Cells | |
|---|---|---|---|
| | (intensity units) | (intensity units) | Ratio Live/Dead |
| Edge of Screw | 17.3 | 4.5 | 3.9 |
| One side of Screw | 69.4 | 37.5 | 1.9 |
| Opposite side of | 43.9 | 28.0 | 1.6 |
| Screw | | | |

In the preceding detailed description, the invention is described with reference to specific exemplary embodiments thereof and locations of use within the spine. Various modifications and changes may be made thereto without departing from the broader spirit and scope of the invention as set forth in the claims. The specification and drawings are, accordingly, to be regarded in an illustrative rather than a restrictive sense.

What is claimed is:

1. A method for therapeutic repair of an injury to bone or tissue comprising:
providing a structural component, wherein the structural component repairs the injury; and
providing a therapeutic fluid, wherein the therapeutic fluid is located within the structural component.

2. The method of claim 1, wherein the therapeutic fluid promotes repair of the injury.

3. The method of claim 1, wherein the therapeutic fluid comprises cells such as mesenchymal stem cells, and proteins, such as fibrinogen and growth factors.
